(19) United States
(12) Patent Application Publication
(10) Pub. No.: US 2022/0022847 A1
(43) Pub. Date: Jan. 27, 2022

(54) ULTRASOUND IMAGING APPARATUS

(71) Applicant: Hitachi, Ltd., Tokyo (JP)

(72) Inventors: Misaki HIROSHIMA, Tokyo (JP); Hiroshi KURIBARA, Tokyo (JP); Kazuaki KAMATA, Tokyo (JP)

(21) Appl. No.: 17/352,696

(22) Filed: Jun. 21, 2021

(51) Int. Cl. A61B 8/08; G01S 15/89; A61B 8/00

(52) U.S. Cl. CPC A61B 8/5207; A61B 8/4472; A61B 8/4488; G01S 15/8918

(57) ABSTRACT

A pitch of transducers is extended, while preventing occurrence of grating lobes. A receive beamformer sets synthetic apertures on an array of transducers that have received echoes of transmission of one transmit beam, and performs processing on received signals of transducers within the receive apertures to form receive beams, followed by performing synthetic aperture processing on the receive beams obtained by the transmission of transmit beams. The positions of the transmit aperture and the receive aperture in the transducer array direction are shifted for every transmission of the transmit beam. A motion vector of the receive aperture along the array direction of the transducers is made different from a motion vector of the transmit aperture so that a distance between phase centers in the successive transmission events is smaller than the case where the transmit aperture and the receive aperture are shifted with a constant vector for each transmission.

### CROSS-REFERENCE TO RELATED APPLICATION

The present application claims priority from Japanese application JP2020-124214, filed on Jul. 21, 2020, the contents of which is hereby incorporated by reference into this application.

### BACKGROUND OF THE INVENTION

### Field of the Invention

The present invention relates to an ultrasound imaging technique that uses ultrasonic waves for imaging the inside of a subject.

### Description of the Related Art

An ultrasound imaging is a technique that uses ultrasound (acoustic waves not intended to be heard, and generally high frequency acoustic waves of 20 kHz or higher) for non-invasively imaging the inside of a subject, including a human body.

The ultrasound imaging is performed according to a technique of forming a transmit beam and a receive beam, referred to as beamforming.

Synthetic aperture imaging is widely used for ultrasonic beamforming. In a typical synthetic aperture imaging, one element (transducer) in an array of elements within a probe transmits an ultrasonic wave, and one or more receiving elements receive reflected signals. Then, transmission and reception are repeated with shifting the positions of the transmission element and one or more receiving elements, sequentially in a direction of the array. The synthetic aperture processing is performed by synthesizing signals received by the receiving elements in different transmission events. In particular, it is referred to as monostatic synthetic aperture (Monostatic SA) for the case of transmitting from one element and receiving by the same one element, and it is referred to as bistatic synthetic aperture (Bi-static SA) for the case of receiving by a plurality of receiving elements. There is another method of synthetic aperture imaging called as synthetic aperture with focused beam (SA with focused beam) where instead of transmitting from one element, a focused beam is transmitted from a transmit aperture comprising a plurality of elements.

In Japanese Unexamined Patent Application Publication No. 2018-110784 (hereinafter, referred to as Patent Document 1), there is disclosed a synthetic aperture technique called as SASB (Synthetic aperture sequential beamforming) method in which a receive aperture comprising a plurality of elements receives reflected signals of a transmitted focus beam, and then, receive beamforming is performed with dividing the beamforming into two stages.

At the first stage, one receiving line passing through a transmit focal point is set along the depth direction, and a receive focal point is set at the same position as the transmit focal point. A received signal of each transducer is provided with a delay amount to form a receive beamform focusing on the receive focal point, and the received signals are delayed with this delay amount, and then added up, whereby a first acoustic line signal is obtained. The acoustic line signal thus obtained is the sum of the reflected signals from a large number of observation points located on a concentric arc centered at the transmit focal point. That is, it is a low-resolution acoustic line signal (referred to as Low Resolution Image (LRI), for example) where the signals at the large number of observation points located on the arc are mixed at an equivalent SN ratio. At the second stage, the first acoustic line signal and other acoustic line signals obtained by shifting the positions of the transmit focal point and the receive focal points, are delayed by a predetermined delay amount, and then they are added up with weighting, whereby a second acoustic line signal is obtained. This delay amount is provided as an amount determined by a distance between the observation points on the acoustic line signal and each transmit focal point. This allows the reflected signals from the observation points to be added up in phase, so that the second acoustic line signal becomes a high-resolution acoustic line signal (referred to as High Resolution Image (HRI), for example). With the processing above, a signal value of each observation point in an observation area is obtained.

In “A new synthetic aperture imaging method using virtual elements on both transmit and receive”, M. Bae, Nam Ouk Kim, Moon Jeong Kang and Sung Jae Kwon, 2015 IEEE, International Ultrasonics Symposium (IUS), Taipei, 2015, pp. 1-4 (hereinafter, referred to as Non-Patent Document 1), there is disclosed a technique that a plurality of receive focusing is set at the positions deviated from the transmit focusing in SASB method. In the first stage, a plurality of the first acoustic line signals is obtained by parallel delay summation processing in one transmission. In the second stage, the acoustic line signals are provided with a delay amount, and weighted summing is performed among a plurality of acoustic line signals obtained by shifting the positions of a virtual transmission element and virtual receiving elements, whereby the acoustic line signals at the observation points are obtained. In this technique, the transmit focusing is regarded as a virtual sound source (virtual transmission element), and a plurality of receive focusing is regarded as virtual receiving elements. The delay amount of the second stage is given according to the distance between the observation point, and the virtual transmission element and the virtual receiving element, thereby aligning phases of the reflected signals from the observation points and adding up the reflected signals, so as to obtain a high-resolution acoustic line signal. Thus, according to the first process and second process in Non-Patent Document 1, the synthetic aperture processing is performed both between the virtual transmission elements and between the virtual receiving elements, to obtain a signal of each of the observation points in the observation area. That is, in the SASB method, not only the acoustic line signal is obtained by the synthesis between transmissions, but also between a plurality of acoustic line signals received in parallel, so that the signal-to-noise ratio of the signals at the observation points can be increased and this improves the resolution.

According to this SASB method, the received signals are combined once in the first stage to make the acoustic line signal, and thus the received signal being combined is transmitted, and a process for obtaining a signal value as to each of the observation points can be performed at the second stage after the transmission of thus combined signal. Accordingly, this produces an implementation advantage that the size of hardware can be reduced. For example, the SASB method is expected to be implemented in a wireless probe or a compact machine that is configured to carry out the first stage within the probe.

Furthermore, in the SASB method described in Non-Patent Document 1, calculation cost and performance can be configured to be scalable according to the number of the receive focusing (=virtual receiving elements), and thus high image quality can be expected, as well as expecting installation in a wide product range.

By the way, in ultrasound imaging, if artifacts (false image) occur, due to physical properties of the ultrasonic wave propagation, and so on, this may hinder accurate inspection and diagnosis. As major artifacts, there are known for example, multiple artifacts caused by repeating multiple reflections in the course of propagation of ultrasonic waves, sidelobe artifacts caused by sidelobes (sub-poles) occurring beside the main lobe (main pole), and grating lobe artifacts caused by strong beam intensity occurring in a direction different from the direction of the main lobe. Among those artifacts, the grating lobe artifacts produce a strong virtual image at a location away from a real image, an array should be designed avoiding the artifacts.

A generation angle θ of the grating lobe is determined by the direction of a main beam, a wavelength λ of an ultrasonic wave, and an element pitch d of the array. For the case of a monostatic (monostatic) array that performs transmitting and receiving on an identical element, when shifting of the position of the transmitting and receiving elements is performed one by one on an element basis, grating lobes occur in the direction of θ that satisfies the following equation where N is an integer:

[Equation 1]

2d sin θ=Nλ

Thus, in order to avoid the grating lobes, the element pitch d of the array is designed to be equal to or less than λ/2 with respect to the wavelength λ.

In Japanese Patent No. 3567039 (hereinafter, referred to as Patent Document 2), there is disclosed a bistatic array where a transmission element array and a receiving element array are separated, having a form where the transmission element array is orthogonal to the receiving element array, and a virtual element is assumed for each combination of a transmission element and a receiving element. In the technique of Patent Document 2, in order to avoid the grating lobes, the virtual elements are unevenly dispersed, and the element arrangement of the transmission element array and the receiving element array is designed so that there is generated an area where a pitch between the virtual elements is smaller than the pitch between the real elements.

It is also known that the grating lobes occur not only according to the arrangement of actual elements but also according to the arrangement of virtual elements, as the case of the transmit focusing and the receive focusing in the SASB.

### SUMMARY OF THE INVENTION

If the element pitch is made smaller in order to avoid the grating lobes, treatment of the array becomes difficult, as well as increasing the number of elements required to maintain a resolution and an S/N. Therefore, there is a problem that cost required for the transducer, wiring, circuits, and signal processing may increase.

It is known that grating lobes occur not only in the real element array, but also in the virtual element array. The grating lobes in the virtual element array may occur as a result of synthesis of multiple transmit and receive beams according to signal processing in synthetic aperture imaging. In order to avoid the grating lobes in the virtual element array, it is necessary to reduce the pitch of virtual elements, as in the case of the real element array.

The positions of the virtual elements in the SASB method described in Non-Patent Document 1 correspond to the positions of the transmit focusing and the receive focusing. If the virtual element pitch is reduced to avoid the grating lobes, the interval between the transmit focal points or between the receive focal points is narrowed, resulting in decrease in the frame rate, increase in the signal processing cost, and so on. Also, the number of virtual elements required to maintain the resolution is increased, which leads to an increase in cost.

An object of the present invention is to extend the pitch of the elements (real elements or virtual elements) with preventing the grating lobes.

An ultrasound imaging apparatus of the present invention includes a transmission element configured to transmit an ultrasonic wave to a subject, a plurality of receiving elements in an array for receiving echoes of the ultrasonic wave, generated in the subject for each transmission event of the ultrasonic wave, a shift controller configured to shift positions of the transmission element and the plurality of receiving elements for each transmission event in a direction of the array, and a receive beamformer configured to synthesize received signals obtained in the plurality of receiving elements for each transmission event, between the plurality of receiving elements and between the transmission events. The shift controller shifts the positions of the transmission element and the receiving elements such that a difference in motion vectors is made different between successive two transmission events, the difference in motion vectors is a difference between a first motion vector representing a shift of the position of the transmission element and a second motion vector representing a shift of the positions of the receiving elements, and the shift of the positions occurring between the transmission event and the previous transmission event.

According to the present invention, even when extending the pitch of the transmission elements and the receiving elements (real elements or virtual elements), it is possible to prevent the grating lobes just by the controlling the shift amount of the elements. Therefore, it is possible to enlarge the synthetic aperture without increasing the number of elements of the transmission elements and the receiving elements. With this configuration, high resolution can be achieved without increasing the cost for device implementation.

### BRIEF DESCRIPTION OF THE DRAWINGS

FIG. 1 is a block diagram illustrating a concept of an ultrasound imaging apparatus according to a first embodiment;

FIGS. 2A to 2C illustrate arrangements of a transmission element and receiving elements and motion vectors of the ultrasound imaging apparatus according to the first embodiment;

FIGS. 3A to 3C illustrate arrangements of a virtual transmission element and virtual receiving elements and motion vectors in SASB method of the ultrasound imaging apparatus according to the first embodiment;

FIG. 4A illustrates positions of the transmission element and the receiving elements and motion vectors for each transmission event of the ultrasound imaging apparatus according to the first embodiment, and FIG. 4B illustrates the positions of the transmission element and the receiving elements and motion vectors for each transmission event according to a comparative example;

FIG. 5A is a graph showing the positions of the phase centers for each transmission event according to the first embodiment, and FIG. 5B is a graph showing the positions of the phase centers for each transmission event according to the comparative example;

FIG. 6 is a block diagram showing a specific configuration of the ultrasound imaging apparatus according to the first embodiment;

FIG. 7 illustrates a transmit beam and receiving beams of the ultrasound imaging apparatus according to the first embodiment;

FIG. 8A illustrates a method for calculating a delay amount used by a first receive beamformer according to the first embodiment, and FIG. 8B illustrates that a signal value of an observation point p of a second receive beamformer is included in the signal value of a representative point Qₙₘ on the receiving line;

FIG. 9 illustrates that a midpoint between the center of the transmit aperture and the center of the receive aperture corresponds the phase center according to the first embodiment;

FIG. 10A illustrates a monostatic array, FIG. 10B illustrates a method of calculating the phase center, and FIG. 10C illustrates that the spacing between the phase centers is regarded as an element pitch d;

FIG. 11 is a flowchart showing a process of a shift controller of the ultrasound imaging apparatus according to the first embodiment, for calculating a shift amount of the receiving elements for each transmission event; and

FIG. 12 is a block diagram of the ultrasonic imager according to a second embodiment.

### DETAILED DESCRIPTION OF THE PREFERRED EMBODIMENTS

There will now be described an ultrasound imaging apparatus according to an embodiment with reference to the accompanying drawings.

### First Embodiment

### Overview

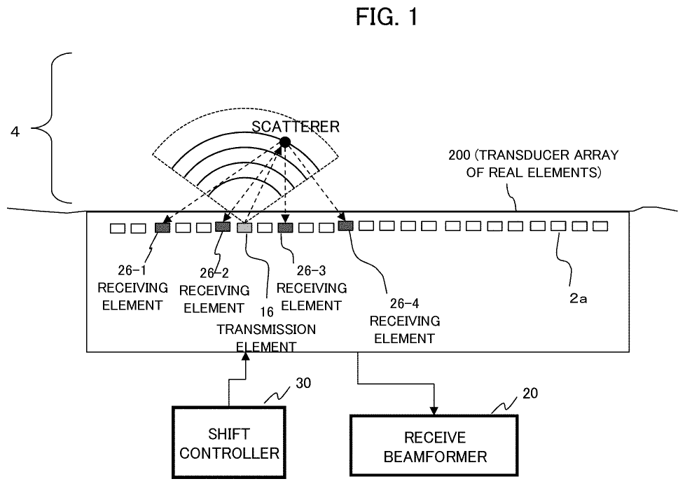

With reference to FIG. 1 and other figures, there will be described the overview of the ultrasound imaging apparatus according to the first embodiment.

As shown in FIG. 1, the ultrasound imaging apparatus of the first embodiment includes a transmission element 16 for transmitting an ultrasonic wave to a subject 4, an array of a plurality of receiving elements 26-1 to 26-4 for receiving echoes of the ultrasonic wave generated in the subject 4 for each transmission event of the ultrasonic wave, a shift controller 30, and a receive beamformer 20.

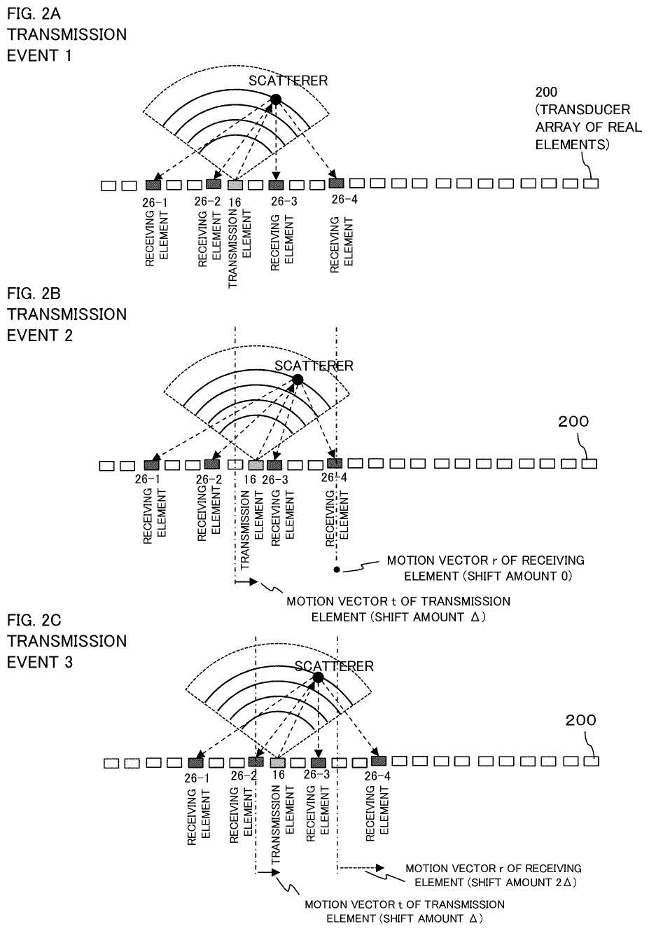

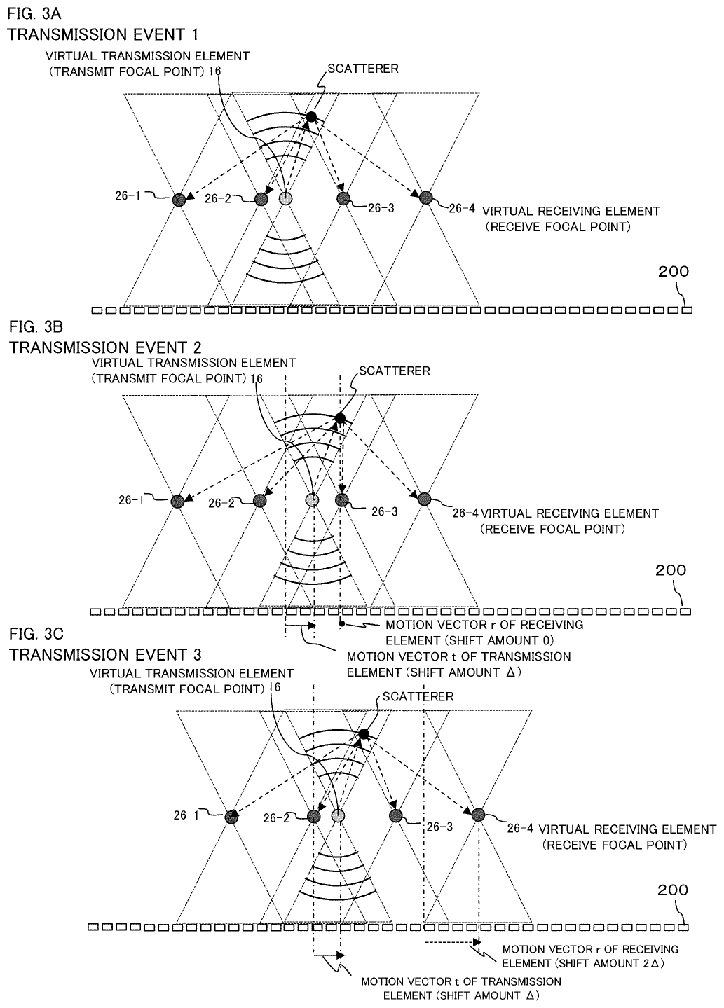

The transmission element 16 and the plurality of receiving elements 26-1 to 26-4 described here may be actual elements (transducers 2a) as shown in FIGS. 2A to 2C, or they may also be virtual elements as shown in FIGS. 3A to 3C (for example, elements assumed to be at the positions of the transmit focusing and the receive focusing in the SASB method as described in Non-Patent Document 1).

For example, as shown in FIGS. 2A to 2C or FIGS. 3A to 3C, the shift controller 30 shifts the positions of the transmission element 16 and the receiving elements 26-1 to 26-4 in the array direction of the receiving elements 26-1 to 26-4, for each transmission event of the ultrasonic wave.

The receive beamformer 20 synthesizes the received signals obtained by the plurality of receiving elements 26-1-26-4 for each transmission event, between the plurality of receiving elements 26-1 to 26-4 and between the transmission events. An ultrasonic image is generated using thus synthesized signals.

As shown in FIGS. 2A to 2C or FIGS. 3A to 3C, a shift of the position of the transmission element 16 between a transmission event and its one previous transmission event is represented as the motion vector t. Similarly, a shift of the receiving elements 26-1 to 26-4 is represented by a motion vector r. The shift controller 30 shifts the positions of the transmission element 16 and the receiving elements 26-1 to 26-4 so that a difference between the motion vector t of the transmission element 16 and the motion vector r of the receiving element 26-1 and others is made different in two successive transmission events.

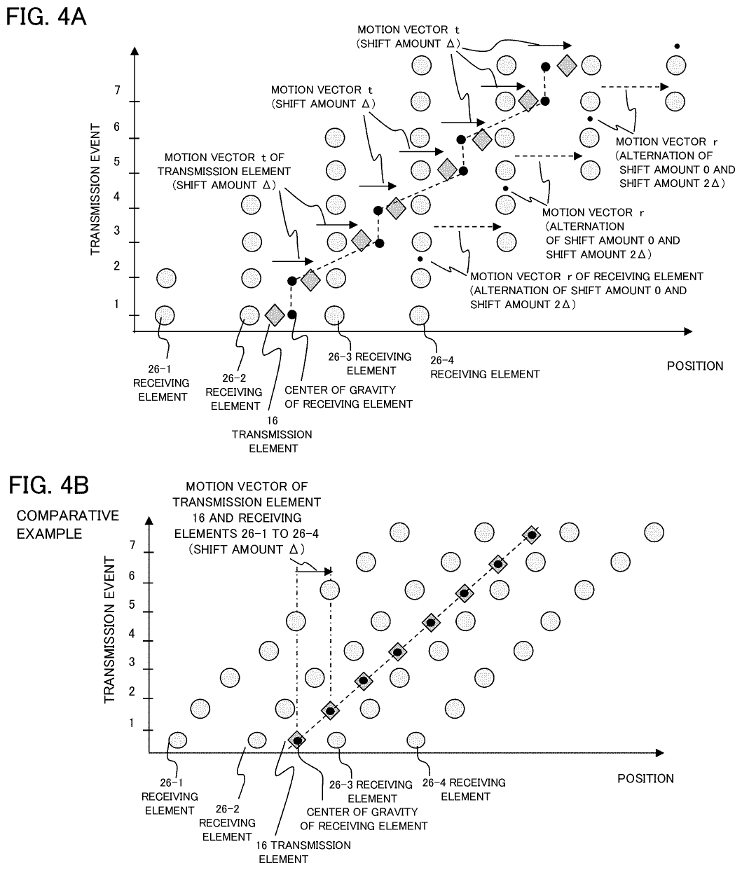

For example, as shown in FIG. 4A, the shift controller 30 is configured to change, for each transmission event, at least either a shift amount or a shift direction of the positions of the receiving elements 26-1 to 26-4. In the example of FIG. 4A, the shift controller 30 alternately sets zero and a predetermined value (for example, 2Δ) as the shift amount of the positions of the receiving elements 26-1 to 26-4 for each transmission event. At this time, the shift controller 30 shifts the position of the transmission element 16 for each transmission event, by a predetermined constant shift amount Δ in a constant direction.

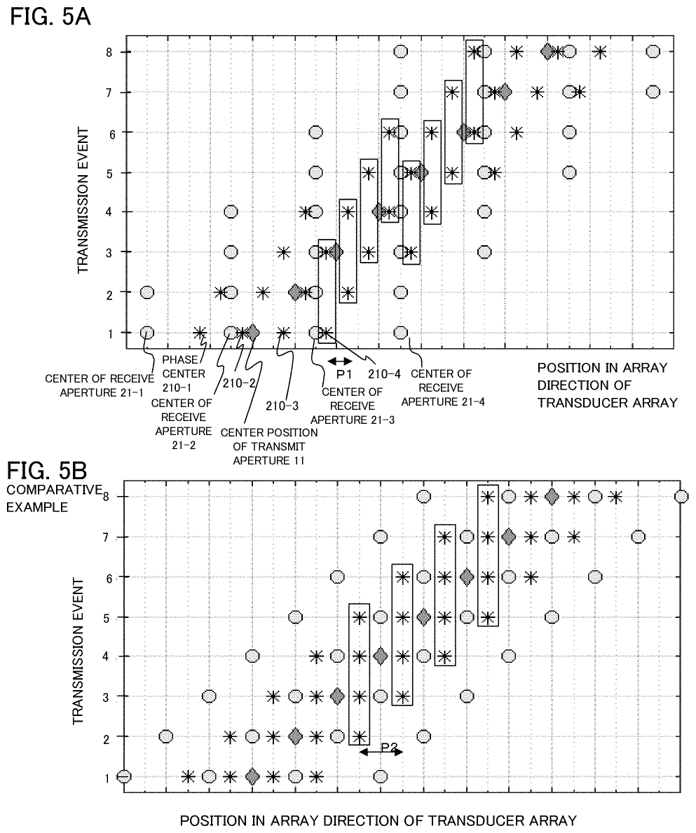

As described above, shifting is performed so that the difference between the motion vector t of the transmission element 16 and the motion vector r of the receiving elements 26-1 to 26-4 is made different in two consecutive transmission events, and phase centers 210-1 to 210-4, which are the midpoints between the transmission element 16 and each of the receiving elements 26-1 to 26-4, are obtained. This allows the distance P1 (FIG. 5A) of the phase centers 210-1 to 210-4 between consecutive transmission events to be smaller than the distance P2 of the comparative example (FIG. 4B and FIG. 5B). In the comparative example, the motion vector t of the transmission element 16 and the motion vector r of the receiving elements 26-1 to 26-4 are made constant for each transmission event (i.e., the motion vector t of the transmission element and the motion vector r of the receiving elements are shifted by a constant amount (shift amount Δ) for each transmission event).

Thus, even when the pitch of the receiving elements 26-1 to 26-4 is made sparser than the comparative example of FIG. 4B and FIG. 5B, it is possible to prevent the grating lobes. Therefore, a high-resolution image can be obtained according to the synthetic aperture processing while exerting control over the amount of data, by using the elements (real elements or virtual elements) with the sparse pitch.

It is desirable the distance P1 of the phase centers 210-1 to 210-4 between successive transmission events should be equal to or less than ½ of the wavelength λ of the ultrasonic wave that is transmitted from the transmission element 16.

It should be noted that the shift controller 30 sets the motion vectors r of the plurality of receiving elements 26-1 to 26-4 to be the same. That is, the receiving elements 26-1 to 26-4 are shifted while maintaining the spacing of the receiving elements 26-1 to 26-4.

Further, the shift controller 30 preferably sets the motion vectors t and r of the transmission element 16 and the receiving elements 26-1 to 26-4 so that the plurality of phase centers 210-1 to 210-4 are set to the positions where the phase centers overlap the same number of times, in repetition of the transmission event.

### Specific Configuration

There will be provided more specific description of the ultrasound imaging apparatus 1 according to the first embodiment.

The ultrasound imaging apparatus 1 described below employs the SASB method to perform the receive beamforming and the synthetic aperture processing, whereby signal values of a plurality of observation points set in the subject are obtained (see FIG. 3B). Thus, as shown in FIGS. 3A to 3C, the transmission element 16 and the plurality of receiving elements 26-1 to 26-4 are virtual elements that are assumed to be at the positions of the transmit focusing and the receive focusing in the SASB method.

However, as already described, the present embodiment is not limited to the SASB method, but it may be any method as long as the method is to calculate the signal values of the observation points from a plurality of received signals according to the bi-static synthetic aperture that performs the synthetic aperture processing both on the transmitting aperture and on the receive aperture (e.g., see FIG. 3A).

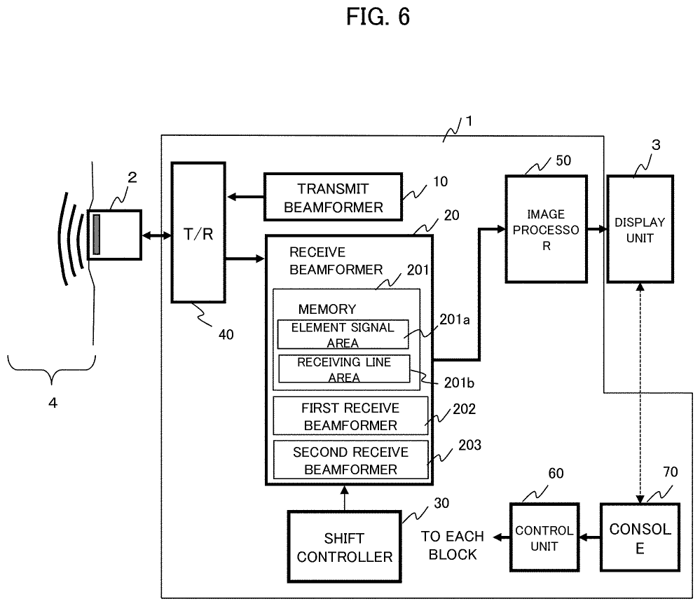

As shown in FIG. 6, the ultrasound imaging apparatus 1 comprises a transmit beamformer 10, a receive beamformer 20, a shift controller 30, a transmission and reception separator 40, an image processor 50, a control unit 60, and a console 70.

The transmit beamformer 10 and the receive beamformer 20 are connected to an ultrasonic probe 2 via the transmission and reception separator 40. The ultrasonic probe 2 incorporates a transducer array 200 in which the transducers 2a capable of transmitting and receiving ultrasonic waves are arranged in a row.

The receive beamformer 20 is provided with a memory 201, a first receive beamformer 202, and a second receive beamformer 203. According to the SASB method, the receive beamformer performs the receive beamforming and the synthetic aperture processing to determine the signal values of a plurality of observation points set in the subject.

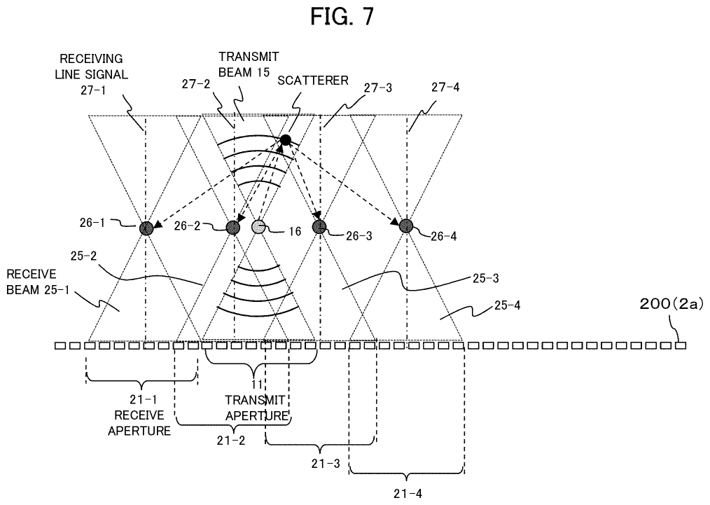

As shown in FIG. 7, the transmit beamformer 10 sets the transmit aperture 11 on the transducer array 200 of the ultrasonic probe 2 and outputs a transmission signal to the transducers 2a in the transmit aperture 11. Each transducer 2a in the transmit aperture 11 converts the transmission signals into an ultrasonic wave, and it is transmitted to the subject 4 as a transmit beam 15. At this time, the transmit beamformer 10 sets a delay time to delay each of the transmission signals to be delivered to the respective transducers 2a, thereby setting the position of the transmit focusing. The position of the transmit focusing becomes the position of the virtual transmission element 16.

As shown in FIG. 7, the transmit beamformer 10 can also transmit as the transmit beam 15, a focused beam that converges to the transmit focusing 16 within the subject 4. Alternatively, a transmit focal point (transmission element) 16 can virtually set on the front side of the transducer array 200 and transmit a beam that spreads within the subject 4 as the transmit beam 15.

A portion of the transmit beam 15 is reflected, scattered, and so on, by a reflector and others in the subject 4, formed as an echo, and reaching the transducer array 200 of the ultrasonic probe 2, and then received by each of the transducers 2a. The received signal outputted from each transducer 2a is temporarily stored in an element signal area 201a within the memory 201 via the transmission and reception separator 40.

The receive beamformer 20 sets a plurality of receive apertures 21-1 to 21-4 on the transducer array 200 for each transmission. In the example of FIG. 7, four receive apertures 21-1 to 21-4 are provided. The receive beamformer 20 reads out the received signals of the plurality of transducers 2a within the receive aperture 21-1, from the element signal area 201a of the memory 201, and performs predetermined processing such as providing a delay amount to each received signal followed by adding up, thereby forming the receive beamform 25-1, calculating a receiving line signal of the receiving line 27-1, and stores the receiving line signal in the receiving line area 201b in the memory 201. A method for calculating the receiving line signal will be described in detail later.

Similarly, for the other receive apertures 21-2 to 21-4, the receive beams 25-2 to 25-4 are formed and the receiving line signals of the receiving lines 27-2 to 27-4 are calculated. Then, the receiving line signals are stored in the receiving line area 201b. Thus, the receiving line signals of the receiving lines 27-1 to 27-4 are stored in the receiving line area 201b for one transmission event.

The shift controller 30 shifts the position of the transmit aperture 11 and the positions of the receive apertures 21-1 to 21-4 in the array direction of the transducer array 200 for each transmission event, thereby shifting the transmit focal point (virtual transmission element) 16 and the receive focal points (virtual receiving elements) 26-1 to 26-4. At this time, the shift controller 30 shifts the positions of the transmission element 16 and the receiving elements 26-1 to 26-4 so that the difference between the motion vector t of the transmission element 16 and the motion vector r of the receiving element 26-1 and others varies in two successive transmission events (see FIGS. 3A to 3C, and 4A).

The receive beamformer 20 performs the synthetic aperture processing on the receiving line signals of the receiving lines 27-1 to 27-4 obtained in more than one transmission event, respectively, within the same transmission event and between the transmission events. This allows calculation of the signal intensity, for example, reflected at the observation points within an observation area that is set in the subject 4.

### Delay Amount in Receive Beamforming

There will now be described in detail the delay amount according to the first receive beamformer 202.

As the first stage of receive beamforming, the first receive beamformer 202 sets the receive apertures 21-1 to 21-4 at a predetermined spacing as shown in FIG. 7. Also, the receive focal points 26-1 to 26-4 of the respective receive beams 25-1 to 25-4 are set at a predetermined depth (here, the same depth as the transmit focal point 16).

The first receive beamformer 202 delays the received signals of the respective transducers 2a in the receive apertures 21-1 to 21-4 by a predetermined delay amount, respectively, and then adds up the signals. In this way, the receiving line signals of the receiving lines 27-1 to 27-4 are calculated.

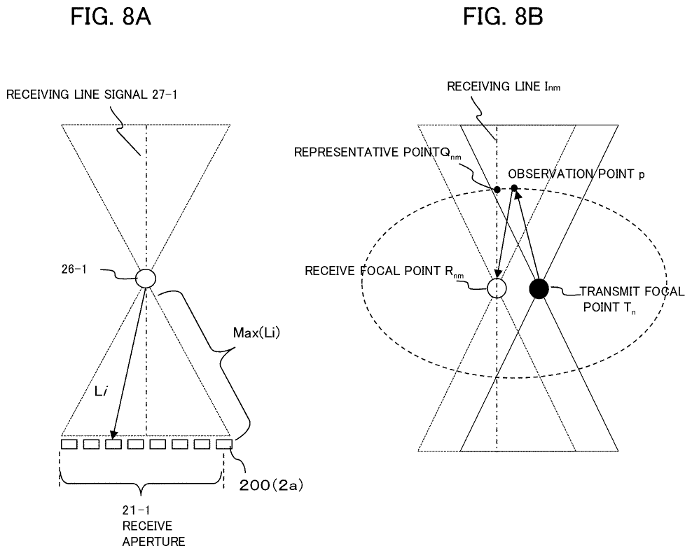

The delay amount for each received signal of the transducers 2a is given by Equation 2. For example, the delay amount Di given to the i-th transducer 2a in the receive aperture 21-1 is calculated by distance Li and sound speed c, between the receive focal point 26-1 and the transducer 2a as shown in FIG. 8A. In Equation 2, max(Li) is the maximum value of the distance between the transducers 2a in the receive aperture 21-1 and the receive focal point 26-1:

[Equation 2]

Di=(max(Li)−Li)/c

It should be noted that the delay amount Di is constant for each transducer 2a, irrespective of the position of the point on the receiving line 27-1 (referred to as a representative point), i.e. the reception time of the received signal.

This delay addition process performed by the first receive beamformer 202 is a process of giving a constant delay amount to each transducer 2a and adding up the received signals outputted from the transducers 2a, and this is implementable by a compact and low-cost analog circuit or digital circuit.

As the second stage, the second receive beamformer 203 performs the synthetic aperture processing on the receiving line signals calculated for the receiving lines 27-1 to 27-4, respectively, between the receive beams and between the transmit beams in a plurality of transmission events. Thus, the intensity of the signals is calculated, which is, for example, reflected at the observation points in the observation area set in the subject 4.

For example, as shown in FIG. 8B, the receiving line signal obtained for the receiving line of Iₙₘ passing through the m-th (m=1 to M) receive focal point Rₙₘ in the n-th (n=1 to N) transmission event, includes the signal value reflected at the observation point p, as the signal value of the representative point Qₙₘ of the receiving line Iₙₘ. The representative point Qₙₘ is the intersection between the receiving line Iₙₘ and the elliptic curve focusing on two points; the transmit focal point Tₙ and the receive focal point Rₙₘ.

Therefore, the second receive beamformer 203 obtains the receiving line signal on the signal of the observation point p, by the synthetic aperture processing according to Equation 3 to obtain the receiving line signal for the receiving line Iₙₘ passing through the first to M-th receive focal point Rₙₘ, for each of the N-th transmission event from the first transmission event.

[Equation 3]

Iₚ=ΣₙΣₘwₙₘ(s)·Iₙₘ(s)

In Equation 3, s represents the position of the representative point Qₙₘ on the receiving line Iₙₘ. Furthermore, wₙₘ represents a weight and it is imparted by the second receive beamformer 203.

For example, the weight wₙₘ can be given using the angle between the central axis of the transmit beam and the point of observation point p, and the angle between the central axis of the receive beam (receiving lines 27-1 through 27-4) and the receive beam.

Thus the second receive beamformer 203 performs the synthetic aperture processing over the interval n between transmissions, and the synthetic aperture processing over the interval m between receptions. This enables obtainment of a high-resolution signal for each observation point, from low-resolution signals combined as the receiving line signal on the receiving line on the first stage.

The image processor 50 converts the signal value of each observation point p in the observation area generated by the receive beamformer 20 into a pixel value of the pixel at a position corresponding to the observation point p, thereby generating an ultrasound image. The generated image is displayed on the display unit 3 which is connected to the image processor 50.

It should be noted the console 70 shown in FIG. 6 receives the imaging conditions from the user.

### Shift Amount of Transmission Element and Receiving Elements

The shift controller 30 controls a shift amount of the transmission element (transmit focal point) 16 and a shift amount of the receiving elements 26-1 to 26-4 for each transmission event, to prevent the occurrence of the grating lobes. Detailed description will be given below.

In the imaging method with the synthetic aperture processing, increasing the synthetic aperture (width of the array of the real elements or virtual elements) can improve the resolution of an image. On the other hand, when the number of elements in the receive aperture (virtual receiving elements or real receiving elements) is increased, a data amount of the received signals (element signals or receiving line signals) is increased, causing an increase of an amount of computation in the receive beamformer 20. To avoid this situation, the receiving element pitch may be made sparse so as not to increase the number of receiving elements, along with extending the receive aperture. However, this may cause grating lobe artifacts.

Therefore, in the present embodiment as described above, the shift controller 30 controls the shift amount of the transmission element (virtual transmission element) 16 and the shift amount of the receiving elements (virtual receiving elements) 26-1 to 26-4, for each transmission event, thereby preventing the occurrence of the grating lobes. Specifically, the shift controller 30 shifts the position of the transmission element and the positions of the receiving elements so that a difference between the motion vector r of the receiving elements and the motion vector t of the transmission element along the array direction of the transducer 2a is made different in two successive transmission events (see FIG. 4A).

In the present embodiment, the receiving elements 26-1 to 26-4 in the same transmission event are arranged at equal spacing, and the motion vector r is made the same in the plurality of receiving elements 26-1 to 26-4. That is, the shift controller shifts the elements while keeping the distance between the receiving elements 26-1 to 26-4.

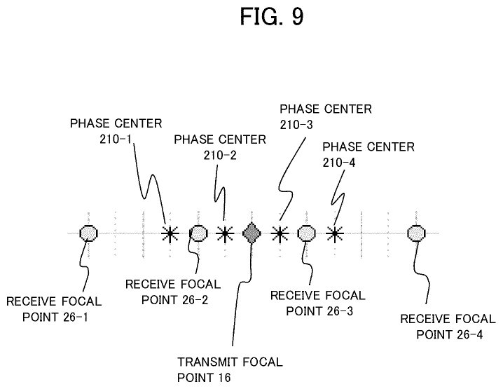

Furthermore, when the phase centers 210-1 to 210-4 are calculated as shown in FIG. 9, the shift controller 30 performs control such that the arrangement interval P1 (FIG. 5A) of the phase centers after the synthesis is performed between the transmission events, becomes equal to or less than ½ of the wavelength λ of the ultrasonic wave received by the transducer array 200.

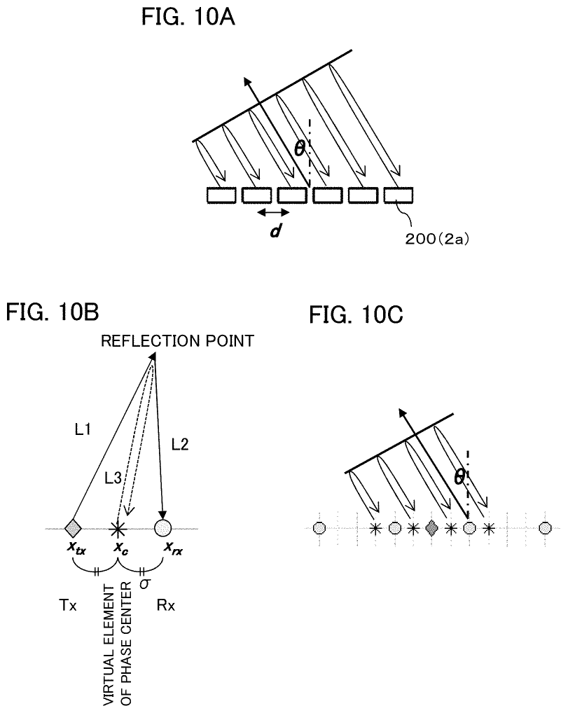

As in FIG. 10A, there is shown the case where transmission and reception at the same transducer 2a are repeated, and the synthetic aperture processing is performed (Monostatic SA). In this case, in general, when a phase difference of a propagation distance between the neighboring elements becomes a multiple of the wavelength, where the propagation distance corresponds to a sum of following propagation distances; the propagation distance of the transmitted wave and the propagation distance of the reflected wave, echo signals of the transducers are intensified mutually and the grating lobes are generated. Therefore, it is known that the spacing d between the elements should be smaller than λ/2 in order to avoid the grating lobes in the monostatic synthetic aperture.

As described in Non-Patent Document 1, the SASB method where a plurality of receive focal points is provided for every transmission, is referred to as the bistatic synthetic aperture, because the transmit focal point 16 is assumed as the virtual transmission element, with the virtual receiving elements (receive focal points) 26-1 to 26-4 being provided, and the transmission element and the receiving elements are different transducers 2a.

Here, as in FIG. 10B, there is assumed a virtual element at the midpoint (phase center) between the transmission element and the receiving element according to a method of the phase center. Then, when the distance from the transmission element to the reflection point is L1, the distance from the reflection point to the receiving element is L2, and the distance from the possible phase-center element to the reflection point is L3, it is known that the following approximate expression is established under the condition that L3 is sufficiently long with respect to the distance c between the phase center and the transmission element or the receiving element:

[Equation 4]

L1+L2=2*L3

This is referred to as the phase center approximation, and the phase center approximation allows the bistatic synthetic aperture to be approximated to the monostatic (mono-static) synthetic aperture where transmission and reception are performed from and to the phase center for every transmission event. Thus, although calculation of the angle at which the grating lobes occur in the bistatic synthetic aperture is more complicated than the monostatic synthetic aperture, it is possible to calculate the angle that generates the grating lobes by a simple equation as described above (Equation 1) according to the phase center approximation. Equation 1 is shown again as the following:

[Equation 1]

2d sin θ=Nλ

That is, as shown in FIGS. 5A and 5B, spacing of the phase centers 210-1 to 210-4 between the transmission events can be considered as the element pitch d in FIG. 10A, and by making this pitch smaller than λ/2, the grating lobes can be avoided (FIG. 10C).

Therefore, in the present embodiment, the shift controller 30 controls the motion vector t of the transmission element 16 and the motion vector r of the receiving elements 26-1 to 26-4 for each transmission event as described above, and shifts positions of the transmission element and the receiving elements so that the difference between the motion vector t and the motion vector r is made different in two successive transmission events (see FIG. 4A). For example, in the specific example shown in FIG. 4A, the shift amount of the motion vector t of the transmission corresponds to the shift amount Δ being the same for each transmission event, whereas the shift amount of the motion vector r of the reception is set to 0 and 2Δ, alternately for each transmission event.

Thus, as a comparative example, when the transmission element 16 and the receiving elements 26-1 to 26-4 are shifted at a constant shift amount Δ for each transmission as in the examples shown in FIGS. 4B and 5B, the arrangement interval P2 of the phase centers between the plurality of transmission events is Δ. On the other hand, when the shift amount of the receiving elements 26-1 to 26-4 is set to be 0 and 2Δ alternately for each transmission event as in FIG. 4A and FIG. 5A of the present embodiment, the arrangement interval P1 (FIG. 5A) of the phase centers between the plurality of transmission events becomes Δ/2, and this is smaller than P2.

Thus, the shift controller 30 controls the shift amount so that this distance P1 becomes ½ or less of the wavelength λ of the ultrasonic wave, thereby preventing the grating lobes.

In general, the shift amount Δ of the transmission element 16 is equal to the pitch of the transducer 2a (real element), and in many cases, the pitch of the transducer 2a (real element) is equal to or smaller than the wavelength λ. In this case, the arrangement interval P1 of the phase centers between the transmission events becomes λ/2 or less, and it is possible to avoid the occurrence of the grating lobe false image.

Therefore, according to the present embodiment, it is possible to provide a large-diameter receive aperture with reducing the amount of data, by using the receiving elements 26-1 to 26-4 at a sparse pitch and small in number. Therefore, it is possible to obtain a high-resolution image by the synthetic aperture processing.

Further, in the present embodiment, the spacing of the receiving elements 26-1 to 26-4 and the number of the receiving elements 26-1 to 26-4 for each transmission are set in advance so that the number of overlapping times of the phase centers 210-1 to 210-4 is the same at each position, as a result of the synthesis in a plurality of transmission events. For example, in FIG. 5A, there are four receiving elements per transmission, and two of the phase centers 210-1 to 210-4 overlap at the same position.

However, in the case where the number of the receiving elements 26-1 to 26-4 is an odd number such as five, variations in the number of overlapping phase centers between the transmission events may occur, for example, two, three, two, and three. Therefore, even if the arrangement interval P1 of the phase centers is ½ or less of the wavelength λ, the grating lobes may occur due to the interval of positions where the number of overlapping phase centers is large. In order to avoid this, in the present embodiment, the number of receiving elements is set in advance so that the number of the overlapping phase centers becomes the same.

### Process for Determining Motion Vectors

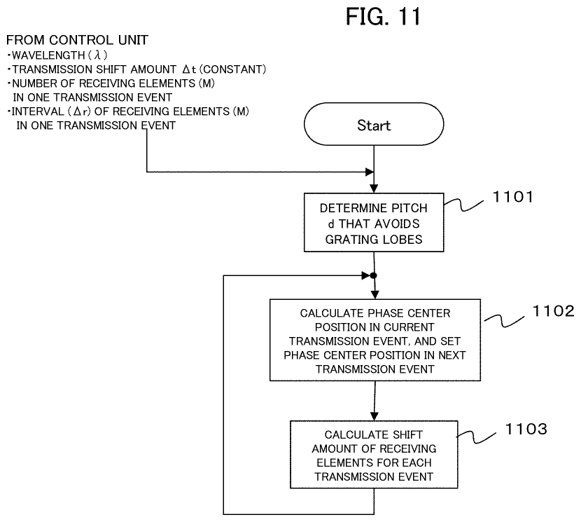

With reference to the flowchart of FIG. 11, there will be described the processing for the shift controller 30 to determine the motion vectors t and r.

In the present embodiment, when the motion vector t of the transmission element 16 is constant, the motion vector r of the receiving elements is made different for each transmission event, thereby avoiding the grating lobes.

The shift controller 30 reads required setting values in advance from the control unit 60, such as the wave length (λ), the shift amount of the transmission element 16 (Δt), the number (M) of the receiving elements 26-1 to 26-M for each transmission, and the interval (Δr) of the receiving elements 26-1 to 26-M in a single transmission event.

First, the pitch d required for avoiding the grating lobes (see FIG. 10A) is determined to ½ of the wavelength λ, for example (step 1101).

Next, the arrangement of the phase centers of the current transmission event is calculated. For example, the transmission element 16 and the receiving elements 26-1 to 26-M of the first transmission event are arranged in order, from one end of the array of the transducers 2a at predetermined intervals, thereby determining the positions of the elements. As shown in FIG. 9, for example, the phase centers 210-1 to 210-M in the first transmission event are calculated from the positions of the determined transmission element 16 and the receiving elements 26-1 to 26-M. The positions of the calculated phase centers 210-1 to 210-M of the first transmission event are shifted by the pitch d, which is previously calculated, in the array direction of the transducers 2a, and then, the positions of the phase centers 210-1 to 210-M in the second transmission event are set (step 1102).

Next, the motion vector r of the receiving elements 26-1 to 26-M is calculated, from the second position of the transmission element 16 which has shifted by the motion vector t (shift amount Δt) from the first position of the transmission element 16, and the positions of the phase centers 210-1 to 210-M in the second transmission event provided in step 1102 (step 1103).

Steps 1102 and 1103 are repeated until the motion vector r of the receiving elements 26-1 to 26-M is calculated for all of the transmission events.

This allows determination of the arrangement of the transmission element 16 and receiving elements 26-1 to 26-M for each transmission event. The shift controller 30 shifts the receiving elements 26-1 to 26-M by the calculated motion vector r, and shifts the transmission element 16 by the constant motion vector t, whereby the arrangement interval of the phase centers between the transmission events becomes d, and the grating lobes can be avoided.

It is alternatively possible that the processing of the flowchart shown in FIG. 11 where the shift controller 30 determines the motion vectors r and t, may be performed for each transmission event, and the motion vectors t and r for shifting the transmission element 16 and the receiving elements 26-1 to 26-M can be determined prior to the subsequent transmission event. Further, the motion vectors t and r may be determined for all transmission events in advance and stored in the memory 201, and the shift controller 30 can read and set the motion vectors t and r, from the memory 201 for each transmission event.

In the present embodiment, the first receive beamformer 202 described above performs the processing to provide a constant delay amount to the received signals and adding the signals. Therefore, it can be implemented by hardware such as a low-cost compact analog circuit or a digital circuit, but it is of course possible that the CPU executes programs stored in the built-in memory to implement the processing by software.

The second receive beamformer 203 and the shift controller 30 can be implemented by software according to the CPU that executes the programs stored in advance in the built-in memory, and it is also possible to configure a part or all of those units by hardware. For example, a custom IC such as ASIC (Application Specific Integrated Circuit) or a programmable IC such as FPGA (Field-Programmable Gate Array) may constitute the second receive beamformer 203 and the shift controller 30, with circuit-designing to implement these functions.

### Second Embodiment

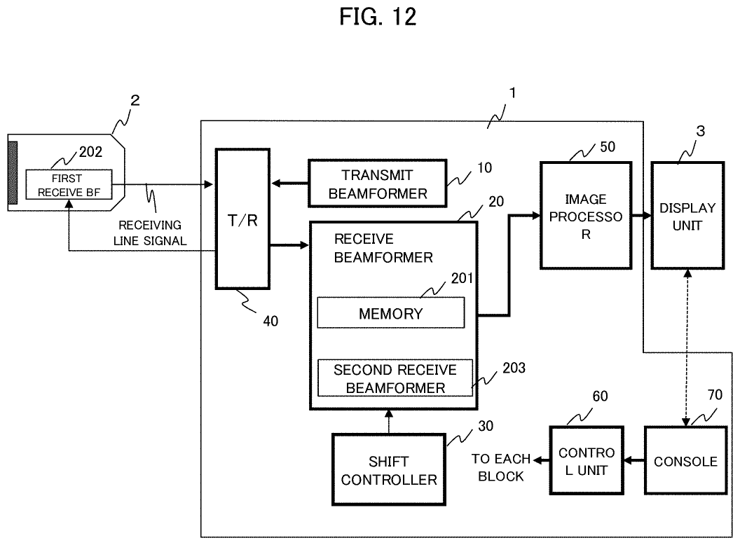

With reference to FIG. 12, there will now be described the ultrasound imaging apparatus of the second embodiment. The ultrasound imaging apparatus of the second embodiment is different from the first embodiment in the point that the first receive beamformer 202 is installed in the probe 2.

Since the other configurations are the same as the apparatus according to the first embodiment, redundant descriptions will not be provided.

The first receive beamformer 202 performs an operation for synthesizing the received signals according to the SASB method to form the receiving line signals, and this operation requires just a small amount of calculation. Therefore, the size of the required arithmetic circuit is also small. Accordingly, this allows installation in the probe 2.

Further, since the number of the combined receiving lines corresponds to the number of the receive apertures, it is sufficient for the probe 2 to transmit to the main body apparatus 1, the receiving line signals the number of which is smaller than the number of the transducers 2a. Therefore, it is possible to reduce the amount of data to be transmitted between the main body apparatus 1 and the probe 2, thereby reducing the scale of the transmission line. This also enables wireless transmission where the amount of data is small.

What is claimed is:

1. An ultrasound imaging apparatus comprising,
a transmission element configured to transmit an ultrasonic wave to a subject,
a plurality of receiving elements in an array for receiving echoes of the ultrasonic wave, generated in the subject for each transmission event of the ultrasonic wave,
a shift controller configured to shift positions of the transmission element and the plurality of receiving elements for each transmission event in a direction of the array, and
a receive beamformer configured to synthesize received signals obtained in the plurality of receiving elements for each transmission event, between the plurality of receiving elements and between the transmission events, wherein
the shift controller shifts the positions of the transmission element and the receiving elements such that a difference in motion vectors is made different between successive two transmission events, the difference in motion vectors is a difference between a first motion vector representing a shift of the position of the transmission element and a second motion vector representing a shift of the positions of the receiving elements, and the shift of the positions occurring between the transmission event and a previous transmission event.

2. The ultrasound imaging apparatus according to claim 1, wherein
the shift controller changes for each transmission event, at least either a shift amount or a shift direction of the positions of the receiving elements.

3. The ultrasound imaging apparatus according to claim 2, wherein
the shift controller shifts the position of the transmission element for each transmission event, by a predetermined constant shift amount in a constant direction.

4. The ultrasound imaging apparatus according to claim 2, wherein
the shift controller alternately sets zero and a predetermined value, as the shift amount of the positions of the plurality of receiving elements for each transmission event.

5. The ultrasound imaging apparatus according to claim 1, wherein
the shift controller sets the first motion vector of the transmission element and the second motion vector of the plurality of receiving elements so that a distance between a plurality of phase centers, being midpoints between the transmission element and each of the plurality of receiving elements, in the successive transmission events, is smaller than the case where the transmission element and the receiving elements are shifted by a constant vector for each transmission.

6. The ultrasound imaging apparatus according to claim 4, wherein
the predetermined value is a shift amount twice as large as the constant shift amount of the transmission element.

7. The ultrasound imaging apparatus according to claim 1, wherein
the shift controller sets the same motion vectors for the plurality of receiving elements.

8. The ultrasound imaging apparatus according to claim 5, wherein
a distance between of the phase centers in the successive transmission events is equal to or less than ½ of wavelength λ of the ultrasonic wave transmitted from the transmission element.

9. The ultrasound imaging apparatus according to claim 5, wherein
the shift controller sets the first motion vectors of the transmission element and the first motion vectors of the receiving elements so that the plurality of phase centers is set to the positions where the phase centers overlap the same number of times, in repetition of the transmission event.

10. The ultrasound imaging apparatus according to claim 1, further comprising a transducer array where transducers are arranged for actually transmitting and receiving the ultrasonic wave, wherein
the transmission element and the plurality of receiving elements are virtual elements that are assumed in SASB (Synthetic aperture sequential beamforming) method,
the transmission element is assumed at a position of a transmit focal point of a transmit beam transmitted from the transducer array to the subject,
the receiving element is assumed at a position of a receive focal point of a receive beam formed by processing received signals by the receive beamformer, the received signals being outputted from the plurality of transducers having received echoes of the transmit beam transmitted from the transducer array, and
the shift controller shifts the transmission element by shifting the transmit focal point of the transmit beam transmitted from the transducer array, and shifts the receiving element by shifting the receive focal point formed by the receive beamformer.

11. The ultrasound imaging apparatus according to claim 10, wherein
the shift controller performs the SASB method to form the receive beam more than one, for each one transmission event of the transmit beam, and performs synthetic aperture processing on the receive beams between the receive beams within the same transmission event and/or between the transmission events.

12. The ultrasound imaging apparatus according to claim 1, further comprising,
an ultrasonic probe incorporating a transducer array where transducers for actually transmitting and receiving the ultrasonic wave, wherein
the receive beamformer comprises a first receive beam former configured to form a plurality of receive beams and a second receive beamformer configured to perform synthetic aperture processing on the receive beams, and
the first receive beamformer is installed in the ultrasonic prove.
